(19) United States
(12) Patent Application Publication
(10) Pub. No.: US 2020/0166459 A1
(43) Pub. Date: May 28, 2020

(54) DIFFERENTIAL FILTRATION SPECTROSCOPIC DETECTION BASED IMMUNOASSAY AND A NOVEL CARTRIDGE FOR DIAGNOSTIC DETECTION OF BIO-PARTICLES

(71) Applicant: KAYA17 INC., Pleasanton, CA (US)

(72) Inventors: SRINAGESH SATYANARAYANA, Pleasanton, CA (US); SULATHA DWARAKANATH, Pleasanton, CA (US)

(21) Appl. No.: 16/690,589

(22) Filed: Nov. 21, 2019

(30) Foreign Application Priority Data
Nov. 22, 2018 (IN) 201841044093

(51) Int. Cl. G01N 21/64; G01N 33/543; B01D 61/14

(52) U.S. Cl. CPC G01N 21/6428; G01N 2021/6439; B01D 61/147; G01N 33/54313

(57) ABSTRACT

The present invention is related to a process of differential filtration spectroscopic detection of bacteria, viruses and biomolecules. The invention also provides a novel cartridge for diagnostic detection of biomolecules/bioparticles.

### RELATED APPLICATION

This application is related and takes priority from the Provisional Application No. 201841044093 filed on Nov. 22^{nd} 2018 with the Indian Patent Office and is incorporated herein in its entirety.

### FIELD OF INVENTION

The present invention is related to a process of differential filtration spectroscopic detection of bacteria, viruses and biomolecules. The invention also provides a novel cartridge for diagnostic detection of biomolecules/bioparticles.

### BACKGROUND OF THE INVENTION

Easy and economical point of care diagnostic tests are important to serve larger and remote communities. To this end, many devices and/method have aimed at designing simple methods to perform diagnostic tests for detection of biomolecules such as bacteria, viruses, proteins or antibodies, that can substitute traditional laboratory tests.

Flow-through tests are generally used in the field for detecting target proteins or microbes. These can be either lateral or vertical flow through processes which vary in the way the antigen-antibody complexes are detected.

U.S. Pat. No. 4,943,522 provides a lateral flow test device for assaying liquid samples that flow laterally across a membrane to be checked in indicator zones. Lateral flow approach is most preferred point of care diagnostic as it can be carried out in easy steps. However, the variation in flow rates between the partitioning membranes may cause differences in the lateral diffusion between samples making it a difficult to use in multiple disease diagnosis. Moreover, lateral flow through processes are time consuming with regard to transfer of test sample laterally from the sample application to the indicator zone, thus being less useful in emergency situations. Thus, flow-through tests are preferred as they are rapid as compared to the lateral flow tests. U.S. Pat. No. 4,818,677 provides a process wherein the analyte is immobilized on a membrane. The test sample is delivered by using an applicator and the detection is done visually which may be error prone. Similarly, U54912034 claims a process wherein the antigen is bound to the membrane. However, here again there is no fluorescence detector involved for the detection.

U55160701 claims a flow-through device wherein the test sample is passed through a reaction matrix containing a capture reagent bound to it to which the target analyte in the test sample may bind. After washing steps, the presence or absence of the target analyte in the test sample is visually determined following the addition of an indicator agent. The process however has many manual steps including washing and rewashing thus being both cumbersome and time-consuming. While U57531362 presents lesser number of steps in describing a flow-through assay, it is still a lengthy process for a point of care test.

The present invention provides a vertical flow-through that is simple and less time consuming as compared to the ones known in the prior art. The key variation in the present flow-through process is that the capture of the analyte is through size separation and not by bonding of the sample to the antigen as found in other prior art.

The present invention also provides a novel cartridge which serves as a substrate for the sample, reagents and the processing mechanism. It also serves as a device to hold the processed sample when it is imaged in the detection chamber. In addition, it also serves as a device that holds the sample during sample preprocessing and for running the assay.

### BRIEF DESCRIPTION OF FIGURES

FIG. 1: Cross Section of the Cartridge (substrate only)

FIG. 2: Cross Section of the Cartridge (Substrate+membrane)

FIG. 3: (a) Alignment guide for alignment of the centers of the cartridge frame and the adhesive (b) Alignment guide for the alignment of the filter membrane to the center of the cartridge frame

FIG. 4: Cross Section of the absorbent pad holder

FIG. 5: Cross Section and Assembly of AB pad holder and Cartridge

FIG. 6: Delivery of the read buffer in the Fluid Mode

FIG. 7: Reader core (detector+excitation source) and cartridge in detection mode

FIG. 8: Block diagram of the reader with cartridge

FIG. 9: Experimental data with detection performed using the cartridge of the invention. The graph provides the detector output with varying amounts of probe.

FIG. 10: Excitation/Emission behavior at the two wavelengths as measured in a spectrophotometer using the same NP-tag sample in a buffer solution

FIG. 11: Cross Section of the closed system Cartridge with top cover and sample well

FIG. 12: Cross Section of the closed system Cartridge with transparent top cover and sample well

FIG. 13. Cross Section and Assembly of AB pad holder and Cartridge with top cover

### DETAILED DESCRIPTION OF THE INVENTION

The present invention provides a vertical flow-through assay process for detecting microbes and biomolecules. In addition, it also provides a novel cartridge that serves as a substrate for processing the sample as well as to hold the processed sample at the time of imaging.

### 1. Vertical Flow-Through Process of the Invention

The present invention provides a vertical flow-through assay process wherein a fluorescently labeled antibody or antigen conjugated to beads or a carrier molecule is combined with sample antigen, antibody or cells, and incubated to form a complex in solution. After an incubation period, this solution is applied to a filter. A wash solution is applied to the top of the filter and passes through. An absorbent pad is placed underneath the filter and captures the wash liquid and unbound fluorescent molecules. The bound contents on the filter are imaged using spectroscopic detection.

It is provided that Vertical flow cartridge process of the present invention has an advantage where the sample travels vertically through the membrane (filter) that is attached to the cavity (also called the detection well) in the cartridge. Thus, vertical flow cartridge can process sample faster than a lateral flow cartridge.

It is also noted that the absorbent pad holder and pads can be used multiple times until they get saturated. Also to be noted is that the pad holder needs to be dried before reuse.

In one aspect, the assay is capable of detecting microbes and/or biomolecules including but not limited to bacteria, viruses, proteins and antibodies.

The samples that are used in the assay are any specimen that is generally used in detecting the disease or condition as envisaged by a clinician. This may include but not limited to human serum or plasma (from blood), saliva, nasal secretions, throat or vaginal swab, urine or stool samples, other body secretions, biopsied tissues, cells in suspension medium or any food sample that can carry the biomolecule or a pathogen. The sample for detection also includes liquid or fluid from non-human specimens such as animal or plant.

The samples to be used in the assay are processed differently for different specimens: for example, whole blood is centrifuged to get serum or plasma; a swab containing the sample is treated with extraction solution. For urine or stool samples, extraction buffer is first used to loosen the sample which is then followed by vortexing and then unwanted material is removed by filtering.

The antigen, as used herein, may have multiple definitions which can be readily identified and appreciated by a person skilled in the art. For example, for bacteria and virus, the antigen is an antibody that is specific to the epitope on the surface of the said bacteria or the virus. For an antibody to be detected, it is a bacteria with an epitope specific to the said antibody. For proteins, it is an antibody that attaches to a specific site on the protein being detected

### Attachment of a Carrier for Size Specificity

A carrier (protein or a polystyrene bead) is attached to the antigen using, for example, an amine-carboxyl linkage. The purpose of this carrier particle or bead or bacteria or nanoparticle is to provide the right size. Size of the carrier particle can be changed based on the antigen size so that the antigen-antibody-carrier-label complex can be held on the filter membrane (described in the following steps). The key variation in the present flow-through process of the invention is that the attachment is based on size separation and not by bonding of the sample to the antigen as found in other prior art.

The antigen-antibody-carrier complex is called the fluorescent probe. It will be appreciated by a person skilled in the art that the present process is designed for fluorescent imaging.

The size of the target bioparticle can be altered to facilitate separation using a nonreactive bead of a suitable size. The filter membrane pore size can be varied so that it can selectively hold the target bioparticle attached to the fluorescent probe.

For example, for a 0.45 μm filter membrane, the fluorescent probe is designed to be 0.4 μm or smaller such as 0.2 or 0.1 μmin size. Beads are usually 1 μm-20 μm or larger. In the case of antibody detection, the antigen is usually a bacteria which may itself be larger than the pore size thus eliminating the need for a carrier. The size of the filter can also be influenced by other particles such as auto-fluorescing particles and large opaque particles in the sample suspension that can interfere with the fluorescence detection of the target.

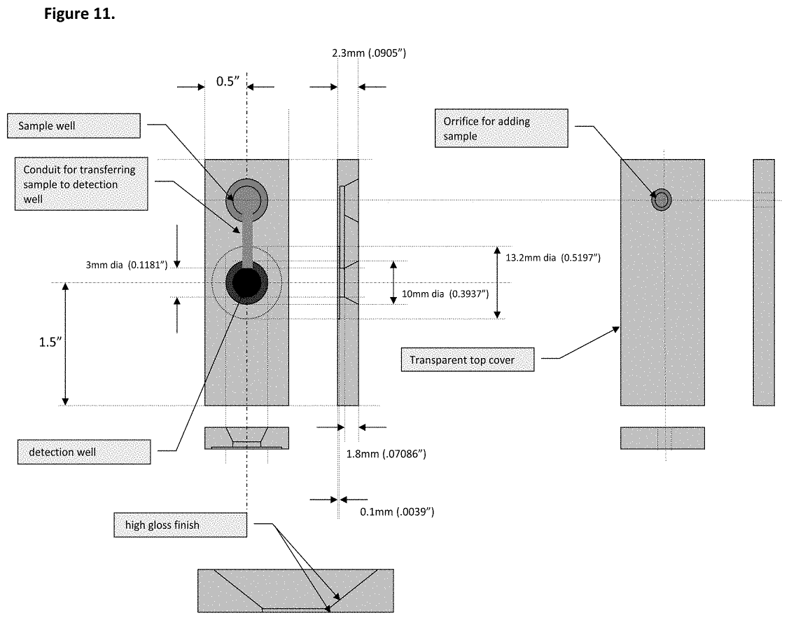

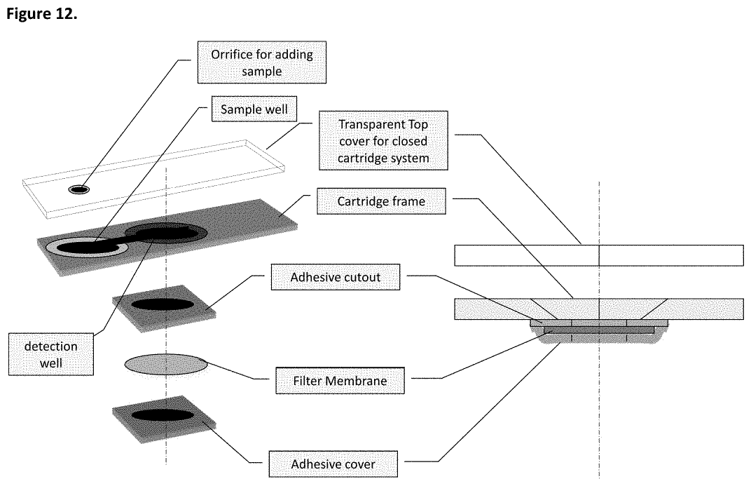

In another embodiment the cartridge design has an additional well that can be added (FIG. 11 and FIG. 12) to the bottom of the cartridge to hold probe solution and the sample. When the combination is left for 5-10 min, the probe attaches itself to the target bioparticle in the sample. The cartridge with the sample and the probe can be vortexed to assist in good mixing of reagents. Once the reaction is complete, the combined liquid can be transferred from the sample well to the detection well. The transfer can happen in two ways.
1. Manually pipetting the liquid and transferring it to the detection well.
2. Forcing the contents of sample well to the detection well using positive or negative pressure through a conduit connecting the sample well and detection well.

The additional sample well can be preloaded with the probe solution and sealed. Before the use of the cartridge the seal can be opened and the sample is delivered to the sample well.

The advantage of having the sample well in addition to the detection well is that it can hold the probe solution preloaded in the cartridge and sealed. It saves the step of transferring the probe from a vial to the detection well. Yet another advantage is that, the reaction between the probe and the sample takes a few minutes and this can happen in the sample well. In the absence of the additional sample well, the reaction has to be done in a separate vial and then transferred to the detection well. If only the bottom of the cartridge is used then the assay is conducted in a “open format”.

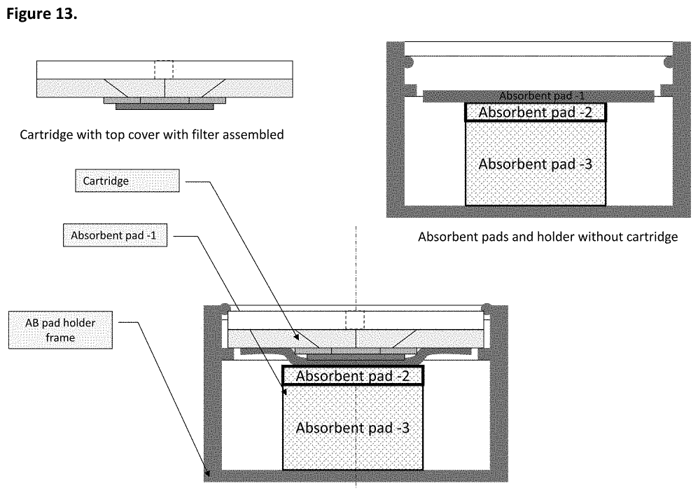

In another embodiment, the cartridge design has a transparent top cover with an orifice whereby the sample and probe can be delivered to the cartridge through the orifice. The use of the top cover converts the system into a “closed” system. The top cover which is transparent prevents contamination of the sample (to be detected for bioparticle) and the probe. Furthermore, the transparent cover allows direct imaging of the detection well through top cover (FIG. 13). Once the sample is delivered to the cartridge through the orifice, it stays inside the cartridge. This prevents any contamination. The transparent top cover allows the user to image the fluorescence through the top cover. The top cover also makes it easy to offer a preloaded probe in the cartridge. An adhesive seal is used to cover the orifice to seal the probe solution until it is used. The orifice can serve a dual purpose of both delivering the sample and feeding positive pressure to push the contents of the sample well to the detection well. The attachment of the top cover and to the bottom of the cartridge can be done by an adhesive. By having an additional sample well and sample preloading in the sample well with the probe (closed system), a separate probe addition step can be avoided.

The detailed assay process is provided herein:

### Attachment of Probe to the Target Bioparticle (Conjugation)

0.5-1 ml of the sample solution is combined with 0.2 ml of the probe in a vial (or the sample well) and left for 5-10 min for the conjugation to happen. The vial (cartridge) is gently shaken or vortexed to mix the two ingredients. Excess quantity of the fluorescent probe is made available so that all the target particles get tagged with probes. The resulting conjugated solution has the following components:
1. Conjugated target (probe attached to target)
2. unattached probes
3. other components of the sample solution and probe solution such as buffer, cells and so on.

### Processing on Cartridge

The cartridge is a 3″×1″ plastic base with a 12 mm diameter hole with a depth of 3 mm in the middle (in the foregoing, the novel cartridge of the invention is described in detail). The filter membrane is attached to the underside of the cartridge. The hole is made with a slanting wall so that the hole with the filter membrane for a well that can hold 1.5 ml-3 ml of liquid. The cartridge is mounted on an absorbent pad stack so that the absorbent stack makes contact to the underside of the filter membrane. In an alternate embodiment, the absorbent membrane can be pre-attached to the underside of the cartridge.

In another embodiment where an additional sample well is also incorporated into the cartridge, a sample of size similar to the detection well is made but there is no hole and filter on the underside for the sample well. The probe is preloaded into the additional sample well and sealed with a removable cover which is removed before use.

The conjugated solution is first poured into the detection well. Some of this liquid starts going through the filter membrane into the absorbent pad. An additional wash buffer 3-5 ml is added to the detection well to flush all the particles in the well that are less than the pore size to go through the membrane and into the absorbent pad. The components left on top of the filter membrane are the conjugated target particles and any other particles that are larger than the pore size. It is usually preferable to do some filtering before the conjugation to remove such large particles in the sample so that they do not interfere with the fluorescence reading.

It is very important that all unattached probes are flushed through the membrane into the absorbent pad. Three passes of 3 ml wash buffers are added to the detection well to ensure that all unattached probes are flushed through the membrane.

Preparation for Fluorescence Reading with Proprietary Reader

What is left on the membrane is now ready for fluorescence measurement. The cartridge is removed from the absorbent membrane assembly and 3 ml of read buffer is added to the well. Since there is no absorbent pad below, the read buffer stays in the well. The read buffer serves two purposes:
1. keeps the conjugated target particles in suspension
2. keep the background fluorescence reading low

The cartridge with the read buffer and the processed sample is then inserted into the reader for reading.

### Detection by Proprietary Reader

The reader is designed specifically to image the whole area of the well (including the walls) so that all the liquid in the well contributed to the reading. Having a wider diameter aids in obtaining a more sensitive measurement.

The whole well area is uniformly excited with the excitation wavelength of the fluorescent probe. The fluorescence emission from the well is passed through an optical filter to isolate only a narrow bandwidth around the emission wavelength.

If a probe with excitation wavelength of 394 nm and emission wavelength of 560 nm is used then a 560 nm filter is used in line with the detector.

The vertical flow cartridge of the invention is used for reflective imaging where the fluorescent probe is chosen such that excitation can vary over a wide range (300 nm-700 nm) and emission can vary from (300-700 nm). However, the excitation and emission are chosen to be distinct. The reader is calibrated apriori to determine the background fluorescence from the cartridge and read buffer. A calibration curve is also generated that maps the fluorescence reading to the number of fluorescent tags in solution.

Based on the reading from the sample, one can determine, using the calibration curve, the number of fluorescent tags and hence the number of target bioparticles in the sample.

### 2. Cartridge of the Invention

The present invention also provides a novel cartridge designed specifically for diagnostic detection of bio-particles

### Construction of the Cartridge

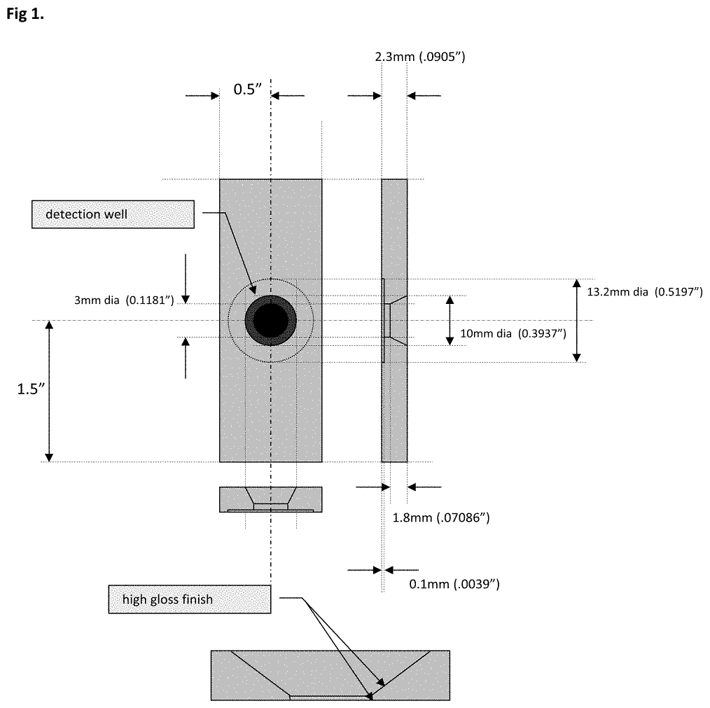

A black non-reflective piece of plastic (metal, ceramic, cellulose, glass can also be used if some special properties are needed), the size of a glass slide 1″×3″ is used. FIG. 1 shows the mechanical drawing of the cartridge in its typical design form.

As can be appreciated, the cartridge of the invention is a substrate for holding the sample, reagents for conducting the assay, the processing mechanism that includes the filter membrane and the absorbent layer. It is also a device that holds the processed sample when it is imaged in the detection chamber.

As an advantage, the cartridge allows the sample (in liquid form) to be spread over an area (usually the cross section of the detection well) so that the processing happens through a broad cross section (unlike conventional LOC) where the sample travels through a thin capillary. This increases the speed of processing thus allowing a shorter time taken for completing the assay. Spreading of the sample over a large area also allows for wide area imaging by a large area photodetector without the need for focusing optics. This is an advantage of the present process described in this invention.

Several materials may be used to fabricate the cartridge and base system of the platform (substrate) such as plastic, glass, ceramics, teflon, silicon. Some exemplary plastics include but not limited to polycarbonate, polyester, polyamide, polyvinyl chloride and polymethyl metacrylate. It is preferred that the materials are nonreactive to the sample, wash buffers and other materials that they are in contact with, thereby maintaining its integrity.

The substrate is about 3 mm thick. However, variations are possible if larger samples are to be processed.

A conical hole is built into the substrate. The top side is wider in opening. The bottom side is smaller. Ratio of diameters is about 2:1. Top-side diameter is about 0.8 inches (20 mm).

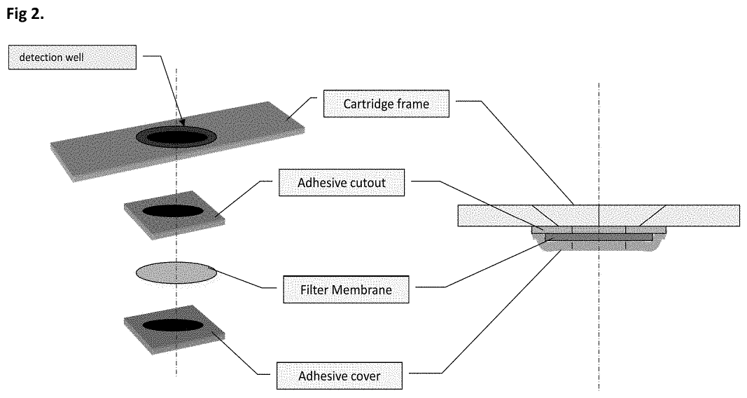

A filter membrane with appropriate pore size is attached to the bottom side of cartridge. Appropriate size, as defined herein, depends on the size of the target bioparticle. For example, if target bioparticle is 0.3 μm then the filter pore should be smaller than 0.3 μm, like for example, 0.25 μm. FIG. 2 shows the attachment of the membrane to the substrate.

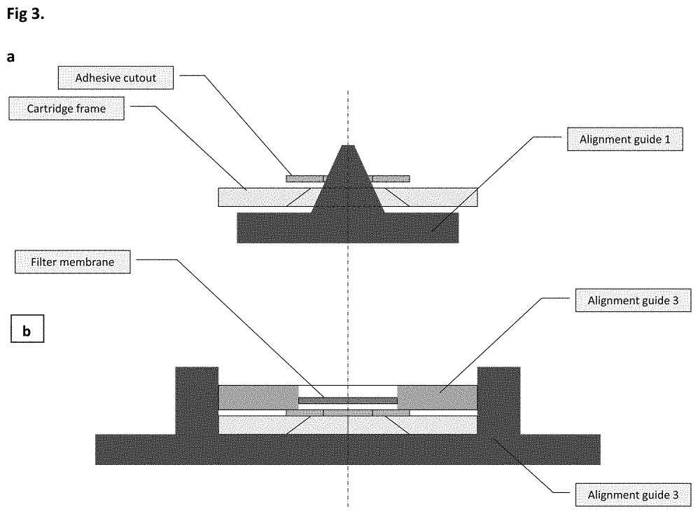

The attachment is done using a double-sided adhesive tape. One side of the adhesive tape bonds to the black substrate plastic and the other bonds to the filter membrane. Filter membrane diameter is larger than the bottom side hole diameter (typically 0.7 inches or 15 mm). The adhesive comes with protective paper on both sides. A rectangular piece is first cut (0.9×0.9 inches or 25 mm×25 mm) and then a hole is punched in the middle of this adhesive paper. A specialized alignment apparatus (FIG. 3) is used to align the center of the hole in the cartridge to the adhesive layer hole and the filter membrane.

The pore size of the cartridge is chosen such that the target sample bioparticle after conjugation with the probe through the assay procedure will not be allowed to pass through. But the unconjugated particles and the unused probe and other particles of no interest pass through the membrane.

The filter membrane itself can be any porous membrane known in the art such as nitrocellulose, mixed cellulose esters, other materials etc. and the dimensions of pores ranges 0.5 μm-0.05 μm and more particularly, 0.45 μm, 0.22 μm, 0.1 μm depending on whether the bioparticle is a protein, bacteria, virus.

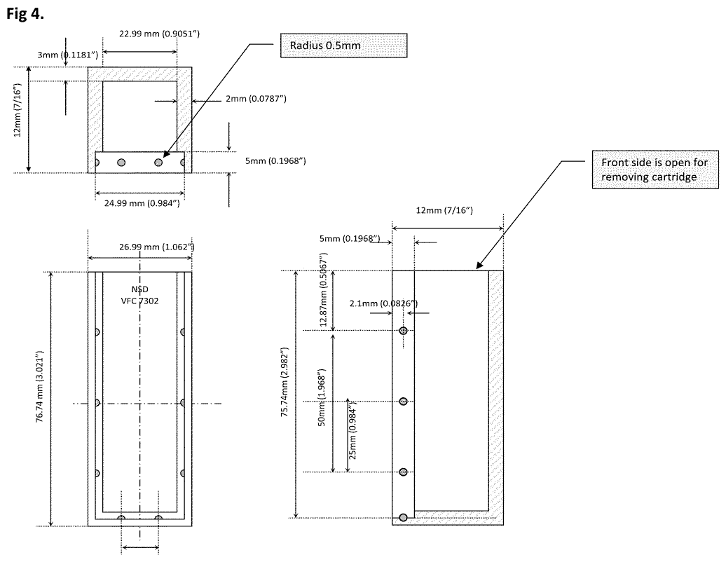

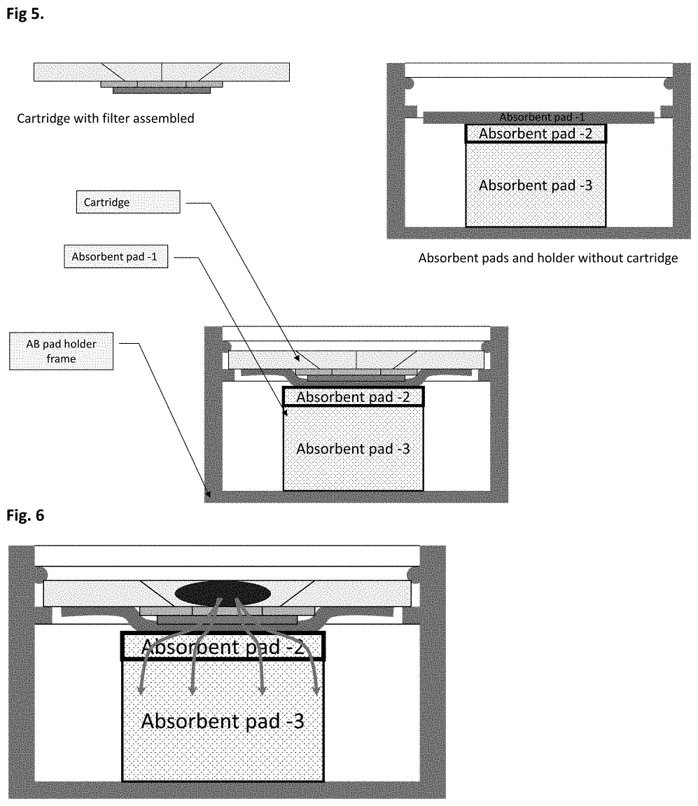

The cartridge is snapped into an absorbent pad holder (FIGS. 4 and 5) during the assay process. The backside of the cartridge, after the filter is attached, makes contact with the absorbent pad when the cartridge is snapped into the holder.

The absorbent pad is typically 1 inch in thickness and made out of the multiple layers of individual absorbent sheets. The absorption properties of the sheets can vary to increase absorption. A small amount of clearance is left below the absorbent pads for bloating when liquid is absorbed.

### Assay of the Invention

The preprocessed sample (typically 5 ml in volume and described under vertical flow-through process) is poured onto the cartridge with the absorbent pad assembly below. Pre-processing can sometimes include cleaning, sediment separation by spinning, coarse filtering, conjugation with the probe. However, sometimes all or parts of the pre-processing can be done on the cartridge.

The assay procedure for the detection of target bio-particle typically comprises the following steps:
a) Fluorescently labeling an antibody, antigen conjugated bead, or a carrier molecule;
b) combining (a) with a sample solution containing a target bioparticle to form a complex in solution;
c) applying the solution from (b) into the top of the detection well of the cartridge of claim 1 and allowing the solution to vertically flow through the filter membrane attached to the bottom surface of the cartridge, and washing the filter membrane from the top with a wash buffer;
d) draining the wash buffer from (c) through the filter membrane using absorbent pad by contacting the absorbent pad to the bottom of the filter membrane and removing the unwanted contents except for the fluorescent probe bound target bioparticle;
e) imaging the bound contents on the filter membrane using spectroscopic detection.

Once the sample is delivered, and after a few minutes, the buffer is absorbed into the absorbent pad (FIG. 6) and the target particles are left on the cartridge. Several wash steps are used to clean out unwanted excess on the filter membrane.

Once all the buffer is drained, the cartridge is removed from absorbent pad assembly and a read buffer is added to the cartridge well. The read buffer quantity is typically 1 ml. This does not drain as there is no absorbent pad underneath.

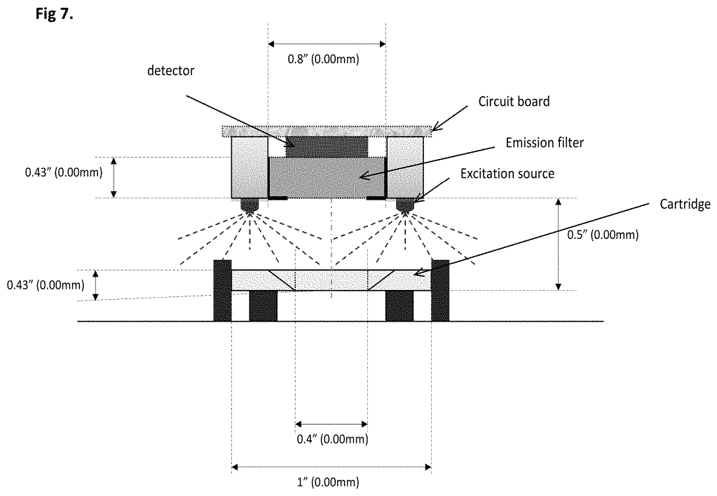

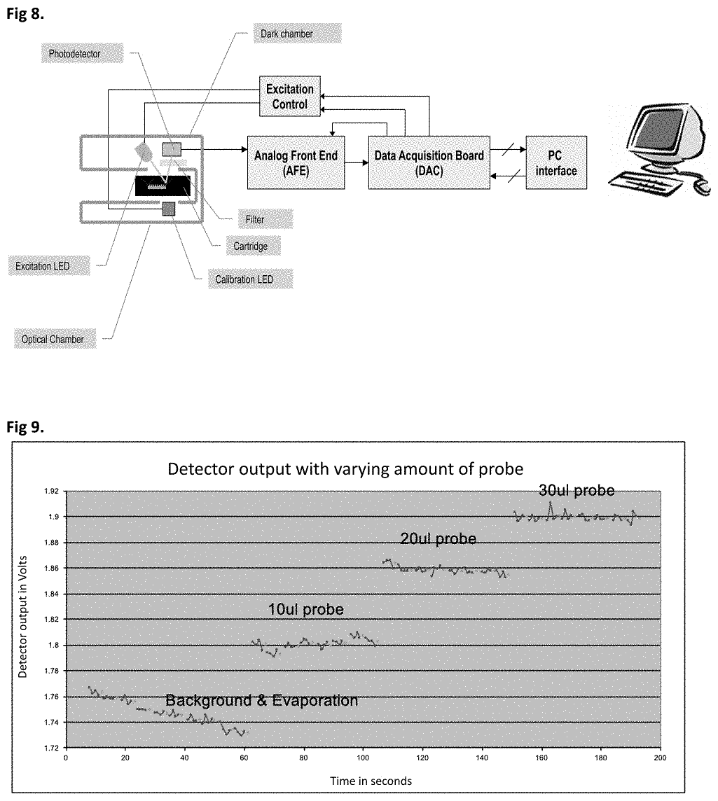

The cartridge is inserted into the diagnostic reader (FIGS. 7 and 8) (fluorescence spectroscopy device). The read buffer is sometimes added after the cartridge is placed in the reader. The diagnostic reader has an excitation source (395 nm) and a detector tuned to detect at the emission frequency (605 nm). An optical filter is used to provide the selectivity. A detector is used to detect the emission from the sample. This signal is amplified and filtered and then used to generate a reading.

Using a pre-calibrated curve the reading is used to determine the concentration of the target bio-particle on the cartridge. Calibration establishes the number of fluorescent probes on an average that get attached to a target bioparticle, and as a consequence the units of light that are emitted by each target bioparticle when excited by a specific strength of excitation.

The reader lid is closed and the device is activated to take a reading. Multiple readings are taken to confirm stability. Read buffer helps in wetting the membrane and decreasing the reflectivity of the membrane. It also prevents the sample from drying up when in the reader. It also helps spread the target molecules around over the detection well to get a higher reading. A reading without target particles but read with buffer only on the cartridge is used for a baseline reference reading.

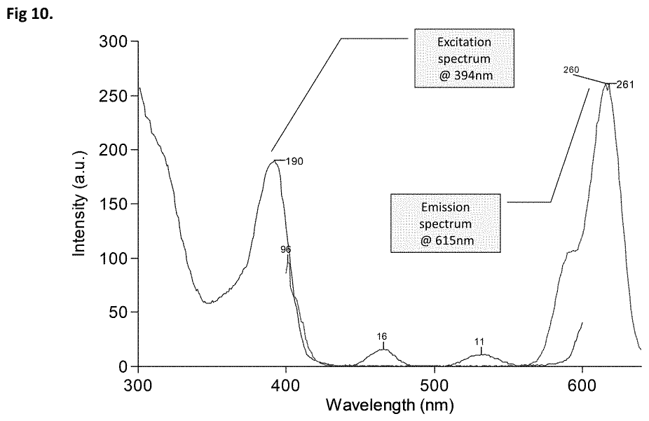

FIG. 9 shows a sample test that was done on the cartridge with increasing amounts of the fluorescent probe tagged to a bio-particle. The readings increase as the concentration of the fluorescent probe increases. FIG. 10 indicates the excitation/emission behavior at the two wavelengths (394 nm and 615 nm) as measured in a spectrophotometer using the same NP-tag sample in a buffer solution.

Cartridge and absorbent pads are disposed after one use. The absorbent pad holder can be reused.

Advantages of the flow-through process and the cartridge of the invention
The flow-through process of the invention involves just three steps:
   Washing of the sample
   Processing the steps of the assay (attach probe and wash off excess
   Detection
The process allows detection over a wide area. Most detection mechanisms are spot detection mechanism where the sample is made to flow through a confined detection spot. Here the sample is intentionally spread over a wide area and a detector that is spread over the sample. Larger sample spread means large detection area using a larger array detector. The sensitivity of the detector can be increased by using larger array detector. There is no need for focusing optics.
By spreading, sample crowding does not take place. If sample is in the form of a drop as opposed to a smear, some light from the particles in the probe that on the bottom do not effectively get to the detector. Assuming detector is to one side of the sample.
Cartridge is very simple to build and hence is low cost. Additional sample well allows to preload the probe thus avoiding the step of transferring the probe from a vial to the detection well.
Absorbent pad and pad holder are reusable.

We claim:

1. A cartridge for diagnostic detection of a target bioparticle present in a sample comprising:
a substrate including a top surface and a bottom surface, and comprising a detection well extending through for receiving a test sample and a probe, which binds specifically to the target bioparticle;
a filter membrane affixed to the bottom surface of the substrate and covering an opening of the well for capturing the target bioparticle bound to the probe, wherein the sample containing the bioparticle travels vertically through the detection well and the filter in the said cartridge.

2. The cartridge as claimed in claim 1, wherein the substrate is a black non-reflective material selected from the group consisting of plastic, metal, ceramic, cellulose, teflon, silicon and glass.

3. The cartridge as claimed in claim 1, wherein the detection well comprises a conical hole with a wider opening at the top surface and a smaller opening at the bottom surface, and wherein the diameter of the filter membrane is larger than the opening at the bottom surface of the detection well.

4. The cartridge as claimed in claim 1, wherein the filter membrane is attached to the bottom of the cartridge using a double-sided adhesive tape with a hole punched, and wherein one side of the adhesive tape bonds to the bottom surface of the substrate and the other side bonds to the filter membrane thereby allowing the test sample to flow through the filter membrane.

5. The cartridge as claimed in claim 4, wherein the hole of the detection well of the cartridge is aligned with the hole on the adhesive tape and the filter membrane.

6. The cartridge as claimed in claim 1, comprising an additional sample well and a top cover, wherein the probe is preloaded into the said additional sample well and sealed with a removable cover, and a reaction is carried out in the said additional sample well after removing the said cover, and the cartridge is configured to transfer the resulting assay sample manually or by positive/negative pressure to the detection well for detection or further processing.

7. The cartridge as claimed in claim 6, wherein the said top cover is transparent comprising an orifice, and wherein the said transparent cover allows imaging of the detection well through the cover.

8. The cartridge for diagnostic detection of target bioparticle present in a sample as claimed in claim 1, wherein the said bioparticle sample is selected from the group consisting of bacteria, bacterial extract, virus, cell or cellular extract, antibody, protein, and combinations thereof.

9. The cartridge for diagnostic detection of bioparticle present in a samples claimed in claim 1, wherein the said sample is selected from the group consisting of human blood, plasma, saliva, nasal secretion, vaginal secretion, saliva, throat swab, urine, stool, biopsied tissue, cells in suspension, fluid from an animal, fluid from a plant and liquid from food sample.

10. The cartridge as claimed in claim 1, wherein the probe is labeled and the label is a fluorescent label.

11. A differential filtration spectroscopic diagnostic detection process for the detection of target bioparticle, wherein the said process is a vertical flow-through process comprising the steps of:
a) Fluorescently labeling an antibody, antigen conjugated bead, or a carrier molecule;
b) combining (a) with a sample solution containing a target bioparticle to form a complex in solution;
c) applying the solution from (b) into the top of the detection well of the cartridge of claim 1 and allowing the solution to vertically flow through the filter membrane attached to the bottom surface of the cartridge, and washing the filter membrane from the top with a wash buffer;
d) draining the wash buffer from (c) through the filter membrane using absorbent pad by contacting the absorbent pad to the bottom of the filter membrane and removing the unwanted contents except for the fluorescent probe bound target bioparticle;
e) imaging the bound contents on the filter membrane using spectroscopic detection.

12. The differential filtration spectroscopic diagnostic detection process as claimed in claim 11, wherein the said target bioparticle is selected from the group consisting of bacteria, virus, antibody and protein.

13. The differential filtration spectroscopic diagnostic detection process as claimed in claim 11, wherein the said sample containing target bioparticle is selected from the group consisting of human blood, plasma, saliva, nasal secretion, vaginal secretion, saliva, throat swab, urine, stool, biopsied tissue, cells in suspension, fluid from an animal, fluid from a plant and liquid from food sample.
